(19) United States
(12) Patent Application Publication
(10) Pub. No.: US 2009/0016991 A1
(43) Pub. Date: Jan. 15, 2009

(54) Rxr Antagonist Treatment Against Multiple Sclerosis

(76) Inventor: Werner Bollag, Basel (CH)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/659,003

(22) PCT Filed: Jul. 16, 2005
(86) PCT No.: PCT/EP2005/007763

§ 371 Date: Jan. 29, 2007

(30) Foreign Application Priority Data
Jul. 29, 2004 (EM) I 04017927.7
Feb. 25, 2005 (EM) I 05004204.3

(51) Int. Cl. A61K 38/21; A61K 31/192; A61K 39/395; A61P 37/02

(52) U.S. Cl. 424/85.6; 514/570; 514/569; 424/85.4; 424/133.1

(57) ABSTRACT

Retinoids with retinoid antagonistic activities, especially Retinoid X Receptor antagonists called RXR antagonists, pharmaceutically acceptable salts and pharmaceutically acceptable esters and amides thereof, have been found to be effective in the treatment of multiple sclerosis, especially by systemic such as oral administration of RXR antagonists.

### CROSS REFERENCE TO RELATED APPLICATIONS

This application is a 371 National Phase Application of PCT/EP2005/007763, filed Jul. 16, 2005, which is incorporated by reference as if fully set forth.

### FIELD OF INVENTION

The present invention relates to the use of retinoid antagonists comprising retinoids with selective Retinoic Acid Receptor (RAR) antagonistic activity, Retinoid X Receptor (RXR) antagonistic activity or mixed RAR-RXR antagonistic activity, for the manufacture of a medicament for the treatment of multiple sclerosis, as well as to the use of such retinoid antagonists for the treatment of multiple sclerosis, to a method of treatment for multiple sclerosis comprising administering such a retinoid antagonist to a patient, to such a retinoid antagonist for use in the treatment of multiple sclerosis and/or to a pharmaceutical composition for use in the treatment of multiple sclerosis comprising such a retinoid antagonist.

### BACKGROUND

Retinoids are a class of compounds structurally related to vitamin A, comprising natural and synthetic compounds. A series of retinoids have been found to be clinically useful in the treatment of dermatological and oncological diseases.

The activity of retinoids is thought to be mediated by the nuclear retinoid receptors RAR α, β, γ and/or RXR α, β, γ belonging to the superfamily of steroid, thyroid hormone, vitamin D, peroxisome proliferator-activated receptors (Pfahl et al., Vitamins and Hormones 49, 327-382 (1994)). Retinoids with receptor agonistic activity bind and activate receptors, whereas retinoids with receptor antagonistic activity bind receptors but do not activate them.

Experimentally, retinoids with retinoid receptor antagonistic activity (retinoid antagonists) are effective in counteracting many properties of retinoids with retinoid receptor agonistic activity (retinoid agonists) such as inhibition of cell proliferation, induction of cell differentiation, induction of apoptosis and inhibition of angiogenesis (Bollag et al., Int. J. Cancer 70, 470-472 (1997). Retinoid antagonists also suppress toxic side effects of retinoid agonists such as the signs and symptoms of the hypervitaminosis A syndrome and teratogenesis (Standeven et al., Toxicol. Appl. Pharmacol. 138, 169-175 (1996); Eckhardt and Schmitt, Toxicol. Letters 70, 299-308 (1994). Therefore, they may be useful clinically in preventing or treating adverse events caused by retinoid agonists.

Retinoid antagonists have been proposed for clinical use in prevention and therapy of retinoid-induced toxicity and side effects, particularly of the so-called hypervitaminosis A syndrome. Retinoid antagonists have also been proposed to be used in combination with retinoid receptor agonists or other nuclear receptor agonists for prevention and treatment of preneoplastic or neoplastic lesions, vitreo-retinopathy and retinal detachment. In addition, retinoid antagonists could be used as single agents, based on their anti-proliferative effect, for treatment of certain neoplasms insensitive to retinoid receptor agonists (see WO 97/09297).

Furthermore, retinoid antagonists have been found to be efficacious in experimental models predictive for the treatment of T-helper cell type 2 (Th2)-mediated immune diseases, or immunoglobulin E (IgE)-mediated diseases, allergic diseases, atopic diseases or diseases mediated by the Th2-related cytokines. They encompass atopic dermatitis (neurodermitis), allergic rhinitis or hay fever and allergic bronchial asthma (see WO 99/24024 and WO 00/53562).

### SUMMARY

For the first time, quite unexpectedly, it has now been found that retinoid antagonists, in particular RXR antagonists, are useful in the treatment of multiple sclerosis, by all kinds of pharmaceutical administration, preferably by systemic, especially enteral, administration.

### DETAILED DESCRIPTION OF THE PREFERRED EMBODIMENTS

In the subsequent detailed specification, wherever the term USE is employed, this refers to the use of retinoid antagonists comprising retinoids with selective Retinoic Acid Receptor (RAR) antagonistic activity, Retinoid X Receptor (RXR) antagonistic activity or mixed RAR-RXR antagonistic activity, for the manufacture of a medicament for the treatment of multiple sclerosis, especially as mentioned as preferred below, the use of such retinoid antagonists for the treatment of multiple sclerosis, to a method of treatment for multiple sclerosis comprising administering such a retinoid antagonist to a patient especially to a patient in need of such treatment in a dose that is effective in said treatment, to such a retinoid antagonist for use in the treatment of multiple sclerosis and/or to a pharmaceutical composition for use in the treatment of multiple sclerosis comprising such a retinoid antagonist preferably in an amount effective in said treatment, if not indicated otherwise.

In the scope and disclosure of the present invention, the term “retinoid antagonists” is used for retinoids or compounds with RAR, preferably RXR or mixed RAR-RXR antagonistic activity.

Besides the other RAR antagonists described in WO 99/24024 and WO 00/53562, which are herewith incorporated by reference with regard to these other compounds and the compound classes mentioned therein, the present invention relates in particular to the USE any one or more of the following compounds:

A compound of the formula I,

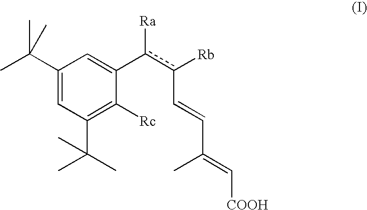

wherein the dotted line represents a bond (thus together with the solid line forming a double bond between the carbon atoms carrying Ra and Rb) or is absent (thus forming a single bond), and when the dotted bond is present, Ra is methyl and Rb is hydrogen, when the dotted bond is absent, Ra and Rb together are methylene thus forming, with the two carbon atoms carrying Ra and Rb, a preferably cis-substituted cyclopropyl ring; and Rc is C₁-C₄-alkoxy; the synthesis of these compounds is disclosed in U.S. Pat. No. 6,326,397;

a compound of the formula II,

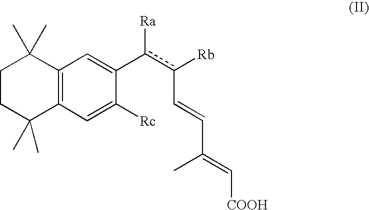

wherein the dotted line represents a bond (thus together with the solid line forming a double bond between the carbon atoms carrying Ra and Rb) or is absent (thus forming a single bond), and when the dotted bond is present, Ra is methyl and Rb is hydrogen, when the dotted bond is absent, Ra and Rb together are methylene thus forming, with the two carbon atoms carrying Ra and Rb, a preferably cis-substituted cyclopropyl ring; and Rc is C₁-C₄-alkoxy; the synthesis of such compounds is described e.g. in L. G. Hamman, J. Org. Chem. 65, 3233 (2000) and SS. Canan Koch et al., J. Med. Chem. 39, 3229 (1996);

or a compound of the formula III,

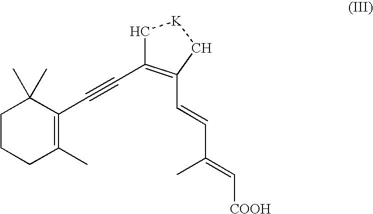

wherein —K— is C₁-C₄-alkylene, especially —CH₂—CH₂—CH₂—, or ═CH—CH═ (thus together with the two carbon atoms binding —K— forming a benzene ring); and Rc is C₁-C₄-alkoxy; the synthesis of such compounds is described e.g. in EP 0 728 742 and U.S. Pat. No. 5,986,131;

or in each case a pharmaceutically acceptable salt thereof, or a pharmaceutically acceptable ester or a pharmaceutically acceptable amide, or in each of the two latter cases a pharmaceutically acceptable salt thereof.

Most preferred is the USE of a compound selected from the group consisting of Retinoid X Receptor (RXR) antagonists compound A, B, C, D, E, F and G listed in Table 1, or a pharmaceutically acceptable salt thereof, especially compound A or a pharmaceutically acceptable salt thereof:

TABLE 1

| Compound | Chemical Name |
|---|---|
| Compound A | (2E,4E,6Z)-7-[2-butoxy-3,5-bis(1,1- |
| | dimethylethyl)phenyl]-3-methyl-2,4,6- |
| | octatrienoic acid |
| Compound B | (2E,4E,)-(1RS,2RS)-5-[2-(3,5-Di-tert-butyl-2- |
| | butoxy-phenyl)-cyclopropyl]-3-methyl-penta-2,4- |
| | dienoic acid |
| Compound C | (2E,4E,)-(1RS,2RS)-5-[2-(3,5-Di-tert-butyl-2- |
| | ethoxy-phenyl)-cyclopropyl]-3-methyl-penta-2,4- |
| | dienoic acid |
| Compound D | (2E,4E,6Z)-7-[3,5-Bis(1,1-dimethylethyl)-2- |
| | ethoxyphenyl]-3-methyl-2,4,6-octatrienoic acid |
| | ethyl ester |
| Compound E | (2E,4E)-3-Methyl-5-[2-(2,6,6-trimethyl-cyclohex- |
| | 1-enylethynyl)-cyclohept-1-enyl]-penta-2,4- |
| | dienoic acid |
| Compound F | (2E,4E)-3-Methyl-5-[(1RS,2RS)-2-(5,5,8,8- |
| | tetramethyl-3-propoxy-5,6,7,8- |
| | tetrahydronaphthalen-2-yl)-cyclopropyl]-penta- |
| | 2,4-dienoic acid |
| Compound G | (2E,4E,6Z)-3-Methyl-7-(5,5,8,8-tetramethyl-3- |
| | propoxy-5,6,7,8-tetrahydro-naphthalen-2-yl)- |
| | octa-2,4,6-trienoic acid |

The expression “pharmaceutically acceptable salts” includes any salt chemically permissible in the art for retinoid antagonists if they bear at least one salt-forming group, e.g. a basic group, such as amino, or especially an acidic group, such as carboxyl or sulfonyl, and that is applicable to warm-blooded animals, especially human beings (e.g. patients), for example in a pharmaceutically acceptable composition. Any conventional pharmaceutically acceptable salt of retinoid antagonists can be utilised. Among the conventional salts which can be made use of, there are the base salts included, for example, alkali metal salts such as the sodium or potassium salt, alkaline earth metal salts such as the calcium or magnesium salt, and ammonium or alkyl ammonium salts.

Where reference is made to a retinoid (e.g. RXR) antagonist within the present disclosure, this refers to the retinoid (e.g. RXR) acid antagonist, an ester or an amide thereof, each in free form and/or in the form of a pharmaceutically acceptable salt (=“a pharmaceutically acceptable amide, ester and/or salt thereof”).

In accordance with this invention, it has been found surprisingly that administration of a retinoid antagonist can be expected to be efficacious in treating patients with multiple sclerosis.

The invention thus especially relates to the USE of a retinoid antagonist (this in a preferred embodiment of the invention relating to a RXR antagonist as mentioned hereinbefore and hereinafter as being preferred) where the disease to be treated is multiple sclerosis that can be treated (=responds to the treatment, be it therapeutically, that is e.g. by alleviation of one or more symptoms and/or by postponement of the development or progress of multiple sclerosis and/or one or more of its symptoms, and/or prophylactically, e.g. by delay or prevention of the start of any stage of multiple sclerosis and/or one or more of its symptoms, such as weakness of the limbs, spasticity, paresthesias, ataxia, tremor, opthalmophlegia, optic neuritis, loss of bladder and bowel control, and/or the like) with such antagonist.

The finding that RXR antagonists (Retinoid X Receptor selective) are useful in treatment of multiple sclerosis is unexpected. Exemplary evidence is presented in this regard in the examples, in a model system for multiple sclerosis (see Example 1).

Multiple Sclerosis (MS) is an inflammatory demyelinating disease of the central nervous system and is considered an autoimmune disease, in particular, a T-helper cell type 1 (Th 1)-mediated immune disease. Pathogenetically, the blood-brain barrier (BBB) is disrupted, followed by migration of auto-reactive blood-derived immune T-cells and macrophages into the brain. This process causes plaque formation in the white matter of brain parenchyma, leading to demyelination of nerve sheaths and axonal loss. These lesions explain the clinical symptomatology of MS.

Matrix-metalloproteinases (MMPs) are enzymes that specifically digest extracellular matrix and basal membrane components. MMP-9 or gelatinase B (EC 3.4.24.35) is considered responsible for the breakdown of the blood-brain barrier, which facilitates the migration of autoreactive T-cells into the brain and the deleterious effects on the central nervous system. This key role of MMP-9 is supported by several facts. In MS, elevated protein levels of MMP-9 are demonstrated in cerebrospinal fluid (CSF) and in serum. Plaque formation, evidenced by magnetic resonance imaging (MRI) scanning, with an appearance of new gadolinium-enhancing lesions occur after an increase of serum MMP-9. Furthermore, MMP-9 mRNA and protein levels in peripheral blood mononuclear cells (PBMC) of progressing MS patients are higher than those of unaffected controls. (See Leppert D et al.: T cell gelatinases mediate basement membrane transmigration in vitro, J Immunol 1995, 154: 4379-4389; Leppert D et al.: Interferon beta-1b inhibits gelatinase secretion and in vitro migration of human T cells: A possible mechanism for treatment efficacy in multiple sclerosis, Ann Neurol 1996, 40: 846-852; Leppert D et al.: Matrix metalloproteinase-9 (gelatinase B) is selectively elevated in CSF during relapses and stable phases of multiple sclerosis, Brain 1998, 121: 2327-2334; Lee M A et al.: Serum gelatinase B, TIMP-1 and TIMP-2 levels in multiple sclerosis. A longitudinal clinical and MRI study, Brain 1999, 122: 191-197; Özenci V et al.: Multiple sclerosis: Pro- and anti-inflammatory cytokines and metalloproteinases are affected differentially by treatment with IFN-β, J. Neuroimmunol. 2000, 108: 236-243; Lindberg R et al.: The expression profile of matrix metalloproteinases (MMPs) and their inhibitors (TIMPs) in lesions and normal appearing white matter of multiple sclerosis, Brain 2001, 124: 1743-1753; Waubant E et al. IFN-β lowers MMP-9/TIMP-1 ratio, which predicts new enhancing lesions in patients with SPMS, Neurology 2003, 60: 52-57; Gelatinase B/matrix metalloproteinase-9 clears interferon-β and is a target for immunotherapy, Brain 2003, 126: 1-11; Gilli F et al.: Neutralizing antibodies against IFN-β in multiple sclerosis: antagonization of IFN-β mediated suppression of MMPs, Brain 2004, 127: 1-10).

All these data indicate that MMP-9 is related to the formation of new MS lesions. Therefore, compounds that inhibit MMP-9 production, release or activity may be expected to have a favourable effect on MS. Interferon-β (IFN-β) has been found to downregulate MMP-9 and this may, in fact, at least contribute to the therapeutic effect of IFN-β in MS.

Surprisingly, it has been found now that RXR antagonists and IFN-β exert their activity in MS by a similar mechanism of action. When MMP-9 expression is measured in peripheral blood mononuclear cells (PBMC), quite unexpectedly RXR antagonists, such as compound A (see Table 1) have a very strong suppressive effect on mRNA and protein expression of MMP-9, measured by ELISA and zymography, for details of a useful assay which can be used generally for any RXR antagonist see Example 1.

In view of this, the invention relates to the USE as described above, including specifically the USE of an RXR antagonist in combination with IFN-β which can lead to a mutually enhancing and even synergistic effect.

The results obtained provide evidence for the suppressive effect of RXR antagonists on the production, release or activity of MMP-9, thus providing evidence for an activity of RXR antagonists in the (prophylactic and therapeutic) treatment of multiple sclerosis (MS). Therefore, the USE of RXR antagonists, especially those mentioned as preferred (see Table 1) in the treatment of this disease is a most preferred embodiment of the present invention. The USE comprises the various stages of MS such as primary progressive MS, relapsing-remitting MS, secondary progressive MS, or acute phase MS, or combinations thereof, and involves therapy as well as prevention of onset and progression.

In addition, a parallel anti-inflammatory property of the RXR antagonists described in European Patent Application 04 017 927.7 which is incorporated by reference herewith in this regard may also contribute to the favourable effects of RXR antagonists in treatment of MS, particularly in stages involving inflammatory processes, e.g. in acute phases, as can also be evidenced with the test system used below in Example 2.

The term “treatment” includes preventive (prophylactic) and/or especially therapeutic treatment. The compounds are being administered in an amount effective to treat that said disease or diseases, especially to a patient in need of such treatment.

For the treatment of MS, the active compound, i.e. a retinoid antagonist, in particular a RXR antagonist, a pharmaceutically acceptable salt, or a pharmaceutically acceptable ester or amide thereof is administered preferably systemically, more preferably enterally, especially orally. Preferably, said active compound is administered as a composition containing said active compound and one or more pharmaceutically acceptable carriers or diluents compatible with said active compound. In preparing such composition, any conventional pharmaceutically acceptable carrier can be utilized. When the drug is administered orally, it is generally administered at regular intervals, conveniently at mealtimes or once daily. Based on information from toxicological studies, the retinoid antagonists are effective in doses which show no or only mild side effects when administered orally. Therefore, oral administration of the active compound is generally preferred.

In the treatment of MS, retinoid antagonists, when administered orally, do not or only slightly induce the adverse events belonging to the toxic syndrome of hypervitaminosis A, such as mucocutaneous, musculoskeletal, neurologic manifestations and elevation of transaminases, triglycerides and cholesterol. In addition, they are less teratogenic in contrast to receptor agonistic retinoids known to be clinically useful in the treatment of dermatological and oncological diseases, such as all-trans retinoic acid (tretinoin), 13-cis retinoic acid (isotretinoin), etretinate and acitretin.

Quite unexpectedly, RXR antagonists, such as compound A (see Table 1) have a very strong suppressive effect especially on protein expression of MMP-9 measured in peripheral blood mononuclear cells (PBMC), determined by ELISA and zymography, see example 1.

In the treatment of MS, retinoid antagonists, pharmaceutically acceptable salts or pharmaceutically acceptable esters or amides thereof, can be used alone or in combination with other treatments, e.g. in combination with one or more other pharmaceutically active substances, preferably pharmaceutically active agents useful in the treatment of multiple sclerosis, such as corticosteroids, interferons, in particular interferon-β, glatarimer acetate (Copaxone®, Teva Marion Partners, TEVA Pharmaceutical Industries Ltd.; a random chain polymer of amino acids Glu, Lys, Ala and Tyr), the antibody pharmaceutical natalizumab (Antegren®, Elan Pharmaceuticals, Inc., and Biogen, Inc.) and/or non-steroidal anti-inflammatory drugs (NSAIDS) that are useful in the treatment of MS.

If used in combination with other substances, retinoid antagonists and said other substances can be administered separately, or incorporated in effective amounts into one pharmaceutical composition, or form a kit of parts the components of which may be administered at separate or overlapping times, preferably such as to allow additional or preferably synergistic efficiency, and/or at the same time.

The aforementioned retinoid antagonists, the salts and esters or amides thereof are especially useful especially in pharmaceutically acceptable enteral, especially oral formulations. These pharmaceutical compositions comprise an active compound in association with a compatible pharmaceutically acceptable carrier material.

Any one or more conventional carrier materials suitable for oral administration can be used. Suitable carriers include water, gelatine, gum arabic, lactose, starch, magnesium stearate, talcum, vegetable oils, polyalkylene-glycols, petroleum jelly and the like. Furthermore, the pharmaceutically active preparations may contain other pharmaceutically active agents. Additionally, additives such as flavouring agents, preservatives, complexing agents, pigments, dyes, stabilizers, tensides, emulsifying agents, wetting agents, solubilizers, buffers and the like may be added in accordance with accepted practices of pharmaceutical compounding.

The pharmaceutical preparations can be made up in any conventional form including inter alia: a solid form for enteral, especially oral administration such as tablets, capsules (e.g. hard or soft gelatine capsules), pills, sachets, powders, granules, or the like. Micronized powders, sprays, aerosols and the like may also be useful, e.g. for administration via the respiratory tract. The pharmaceutical preparations may be sterilized and/or may contain adjuvants such as preservatives, stabilizers, wetting agents, emulsifiers, salts for varying the osmotic pressure and/or buffers.

An example for a preferred oral dosage form comprises tablets, pills, sachets, or capsules of hard or soft gelatine, methylcellulose or of another suitable material easily dissolved in the digestive tract. Each tablet, pill, sachet or capsule can preferably contain from about 10 to about 500 mg, more preferably from about 20 to about 200 mg, of active ingredient. The active ingredient may, based on the weight of the complete oral dosage form, be present in an amount from 2 to 98% by weight, preferably from 20 to 90% by weight. The oral dosages contemplated in accordance with the present invention can vary in accordance with the needs of the individual patient (e.g. the condition of the patient, the size, the age, possible interferences with other therapeutic measures and the like) as determined by the prescribing physician. Generally, however, a daily dosage of from 0.2 to 20 mg per kg of body weight, preferably 0.5 to 10 mg, and most preferably from about 1 mg to about 3 mg per kg of body weight of the patient is utilized. This dosage may be administered according to any dosage schedule determined by the physician in accordance with the requirements of the patient.

The dosage for treatment typically depends on the route of administration, the age, weight and disease condition of the individual. Suitable dosage forms are known in the art or can be easily obtained in a manner known per se. Formulations of hard or soft gelatine capsules, tablets and sachets that are particularly suitable in the scope of the present invention can be easily adjusted in accordance with the above teaching in the art.

The pharmacological activity of the retinoid antagonists as disclosed above may be demonstrated in various test models as shown below, using especially the compounds: A, B, C, D, E, F and G, listed in Table 1.

Besides these mentioned dosage forms, also parenteral dosage forms (e.g. solutions or dispersions for injection and/or infusion) are envisable as useful in the invention, however, enteral treatment and the corresponding dosage forms appropriate for enteral administration are preferred.

Some preferred embodiments of the invention are comprised in the claims, especially the dependent claims, all of which are included here by reference, and in the examples.

The following examples serve to illustrate the invention without limiting its scope:

### EXAMPLE 1

### Effect of RXR Antagonists in a Model System for Multiple Sclerosis (MS), Based on the Key Role of Matrix Metalloproteinase MMP-9 in the Pathogenesis of MS

For details on the method see: (A) Effect of RXR antagonists on expression of MMP-9 protein in human peripheral blood mononuclear cells (PBMC) in vitro, Leppert D et al, J Immunol 1995, 154:4379-4389; (B) Leppert D et al, Ann Neurol 1996, 40: 846-852; (C) Leppert D et al, Brain 1998, 121, 2327-2334; (D) Lindberg R et al, Brain 2001, 124: 1743-1753; (E) Gilli F et al, Brain 2004, 127: 1-10, which are incorporated by reference herein, especially with regard to the method.

Method: Fresh human peripheral blood mononuclear cells (PBMC) were prepared and expanded by stimulation with PHA as described in Leppert D et al, Ann Neurol 1996; 40: 846-852. 1×10⁶ cells were pretreated with the RXR antagonist compound A in concentrations of 10⁻⁶ M, 10⁻⁷ M and 10⁻⁸ M for 18 hours. Vehicle was used as control.

In two different experiments, after pretreatment with compound A, PBMCs were stimulated for 24 hours with Interleukin-2 (IL-2) 50 U/ml in one group, or not stimulated with IL-2 in another group.

The effect of the mentioned concentrations of compound A was compared with vehicle controls in the IL-2 stimulated and in the non IL-2 stimulated cultures of PBMC. The expression of MMP-9 protein was determined by analysis with zymography and ELISA, see especially references (A) under “Zymographic analysis of conditioned medium (CM)” and (C) under “ELISA (enzyme linked immunosorbent assay) for MMP-9” mentioned above, respectively.

Results As can be seen from Tables 2 and 3, the RXR antagonist compound A had a marked suppressive or inhibitory effect on the production, release and/or activity of MMP-9. The degree of inhibition of expression of MMP-9 protein was dose dependent and could be induced in IL-2 stimulated and in not IL-2 stimulated cultures of PBMCs.

TABLE 2
Effect of different concentrations of RXR antagonist compound A
on MMP-9 protein expression in PBMCs not stimulated with
IL-2-results given as MMP-9 total protein in the supernatant in
ng/ml (left) and % inhibition of MMP-9 protein expression compared
with the vehicle control (0%) (right).

MMP-9

| Dose/Concentration | | Inhibition of |
|---|---|---|
| compound A | Protein (ng/ml) | expression (%) |
| Vehicle control (0 M) | 71 | 0 |
| 10⁻⁶ M | 33 | 53 |
| 10⁻⁷ M | 43 | 39 |
| 10⁻⁸ M | 51 | 28 |

TABLE 3
Effect of different concentrations of RXR antagonist compound A
on MMP-9 protein expression in PBMUs stimulated with
IL-2-results given as MMP-9 total protein in the supernatant in
ng/ml (left) and % inhibition of MMP-9 protein expression compared
with the vehicle control (0%) (right).

MMP-9

| Dose/Concentration | | Inhibition of |
|---|---|---|
| compound A | Protein (ng/ml) | expression (%) |
| Vehicle control (0 M) | 61 | 0 |
| 10⁻⁶ M | 29 | 52 |
| 10⁻⁷ M | 41 | 33 |
| 10⁻⁸ M | 40 | 34 |

Conclusions: RXR antagonists inhibit MMP-9 production, release and/or activity in a pharmacological model system for multiple sclerosis. Since MMP-9 activity is considered a responsible key factor in the pathogenesis of multiple sclerosis, RXR antagonists are expected to be useful in the therapy of MS and in the prevention of progression of the clinical manifestations of multiple sclerosis. Therapy and prevention of MS with RXR antagonists is useful in the various stages of MS: primary progressive MS, acute phases, relapsing-remitting MS and/or secondary progressive MS.

The following example shows an additional beneficial anti-inflammatory effect in the treatment of inflammatory diseases that may support the use of retinoid antagonists in the treatment of multiple sclerosis which is an inflammatory demyelinating disease of the central nervous system:

### EXAMPLE 2

### Example for Inflammatory Diseases of Bones and Joints: Effect of RXR Antagonists on Degradation/Destruction of Human Cartilage Induced by Synovial Fibroblasts Taken from Patients with Rheumatoid Arthritis. Ex Vivo, In Vitro Model System for Rheumatoid Arthritis (RA) and Osteoarthritis (OA)

Methods: The effect of RXR antagonists on the activity of synovial fibroblasts, dependent on their state of activation, i.e. modified by a concomitant stimulation by the inflammatory cytokine Interleukin-1β (IL-1β), was determined. Furthermore, it was determined whether this was accompanied by a modulation in the accumulation of the mRNA encoding catabolic enzyme matrix metalloproteinase-1 (MMP-1), responsible for degradation of human cartilage and consequently joint destruction in man. Adherent synovial fluid cells taken from a patient with RA were used after 5 passages in an in vitro assay for cartilage destruction. The cells incubated in flasks coated with 0.1% (0.1 g/100 ml) human cartilage powder were fixed using Matrigel® (BD Biosciences, Becton, Dickinson & Co., Boston, Mass., USA). The release of sulphated glycosaminoglycan (sGAG) into the culture medium was monitored by a commercial colorimetric test according to a method described by S. Björnsson, see Anal. Biochem. 256, 229-237 (1998) using an alcian blue dot plot analysis, and the accumulation of mRNA encoding MMP-1 was quantified by real time PCR (TaqMan® (Roche Diagnostics, Basle, Switzerland)).

The retinoid agonists all-trans retinoic acid and 9-cis retinoic acid, both physiological metabolites of vitamin A, as well as the RXR antagonist compound A, diluted first in ethanol, and then diluted with vehicle or medium to the desired dose/concentration were tested in a time course (0-35 days for the in vitro assay, 0-48 hours for MMP-1 mRNA, see tables 7, 8 and 10) and dose-dependent (10⁻⁷ to 10⁻⁹ M, see tables 5, 6 and 9). This was conducted in the presence or absence of IL-1β (100 pg/ml).

Results: In the absence of IL-1β, the retinoid pan agonist 9-cis RA increased cartilage destruction in vitro in a dose-dependent manner (maximal between 10⁻⁷ M and 10⁻⁸ M), whereas the RXR antagonist compound A, in contrast, had no effect on the basal activity of synovial fibroblast (Table 4).

TABLE 4
In vitro cartilage degradation. Dose dependency.
Effect of 9-cis retinoic acid (9-cis RA) versus compound A
(RXR antagonist) in absence of IL-1β. Release of sGAG in
μg/ml/14 days.

sGAG in μg/ml

| Dose/Concentration | 9-cis RA | compound A |
|---|---|---|
| Vehicle control | 48 | 48 |
| 10⁻⁹ M | 64 | 46 |
| 10⁻⁸ M | 107 | 57 |
| 10⁻⁷ M | 189 | 39 |

However in the presence of IL-1β, quite surprisingly, the RXR antagonist compound A markedly inhibited the IL-1β dependent cartilage destruction, evidenced by a decrease in sGAG (Table 5).

TABLE 5
In vitro cartilage degradation. Dose dependency.
Effect of 9-cis RA versus compound A (RXR antagonist) in
presence of 100 pg/ml IL-1β. Release of sGAG in μg/ml/14 days.

sGAG in μg/ml

| Dose/Concentration | 9-cis RA | compound A |
|---|---|---|
| Vehicle control | 173 | 173 |
| 10⁻⁹ M | 204 | 144 |
| 10⁻⁸ M | 189 | 89 |
| 10⁻⁷ M | 221 | 41 |

The time course confirms that the retinoid agonist 9-cis RA markedly increased cartilage destruction in vitro, whereas with the retinoid antagonist compound A this was not the case. This effect is observed both in the presence and absence of IL-1β (Tables 6 and 7):

TABLE 6
In vitro cartilage degradation. Time dependency.
Effect of 9-cis RA versus compound A (RXR antagonist)
in absence of IL-1β.
Release of sGAG in μg/ml/14 days.

| | Cumulative μg/ml sGAG/14 days | | |
|---|---|---|---|
| | | 9-cis RA | compound A |
| Days | Control | 10⁻⁸ M | 10⁻⁸ M |
| 7 | 23 | 44 | 44 |
| 14 | 48 | 107 | 57 |
| 21 | 84 | 206 | 57 |
| 28 | 112 | 253 | 39 |
| 35 | 117 | 292 | 34 |

TABLE 7
In vitro cartilage degradation. Time dependency.
Effect of 9-cis RA versus compound A (RXR antagonist) in
presence of 100 pg/ml IL-1β. Release of sGAG in μg/ml/14 days.

| | Cumulative μg/ml sGAG/14 days | | |
|---|---|---|---|
| | | 9-cis RA | compound A |
| Days | Control | 10⁻⁸ M | 10⁻⁸ M |
| 7 | 86 | 68 | 67 |
| 14 | 173 | 189 | 89 |
| 21 | 212 | 330 | 89 |
| 28 | 249 | 407 | 173 |
| 35 | 271 | 441 | 56 |

Finally, the cartilage destruction in vitro correlated well with the accumulation of MMP-1 mRNA in synovial fibroblasts incubated for 12 hours. (Tables 8, 9):

TABLE 8
Matrix metalloproteinase-1 (MMP-1) production. Dose dependency.
Effect of 9-cis RA versus compound A (RXR antagonist) MMP-1
mRNA (real time PCR, fold increase of baseline value, after 24
hours) in relative units.

MMP-1 mRNA in
relative units

| Dose/Concentration | 9-cis RA | compound A |
|---|---|---|
| Vehicle control | 1 | 1 |
| 10⁻⁹ M | 1.54 | 1.05 |
| 10⁻⁸ M | 3.39 | 1.12 |
| 10⁻⁷ M | 2.60 | 0.68 |

TABLE 9
Matrix metalloproteinase-1 (MMP-1) production. Time dependency.
Effect of 9-cis RA versus compound A (RXR antagonist) MMP-1
mRNA (real time PCR, fold increase of baseline value, after 0 to
24 hours) in relative units.

| | MMP-1 mRNA in relative units | | |
|---|---|---|---|
| | | 9-cis RA | compound A |
| Hours | Control | 10⁻⁸ M | 10⁻⁸ M |
| 0 | 1 | 1 | 1 |
| 2 | | 0.98 | 1.13 |
| 6 | | 1.31 | 0.81 |
| 12 | 1.13 | 3.39 | 1.12 |
| 24 | | 3.79 | 0.87 |
| 48 | 1.09 | 1.47 | 0.83 |

Conclusion: RXR antagonists inhibit cartilage destruction in a pharmacological model system for destruction of joints in rheumatoid arthritis and osteoarthritis and are thus effective against inflammatory diseases.

The following Examples exemplify pharmaceutical formulations for treating MS by oral administration of RXR antagonists. The formulations mentioned therein are useful for USE in the present invention and were prepared according to the tables presented and using standard procedures, where “Active compound” stands for any one of compounds A, B, C, D, E, F and G mentioned in Table 1, preferably for compound A:

### EXAMPLE 3

### Fill Mass for Soft Gelatin Capsules and Capsules Filled with said Fill Mass

A fill mass for soft gel capsules was prepared using the following components:

TABLE 10
a) Fill mass for soft gelatin capsules

| | | |
|---|---|---|
| Active compound | 10-200 | g |
| Oil* | 1-3 | parts |
| Wax mixture** | 1-5 | parts |
| Fill volume | 1-6 | minims |

*natural vegetable oils, e.g. soy oil, peanut oil, and artificial glycerides
**composition of natural and artificial waxes or partially hydrogenated fats

This fill mass was then used to produce soft gelatine capsules with the following content:

TABLE 11
b) Soft gelatine capsules containing 20-100 mg active substance
20 mg soft gelatine capsule

| Ingredients | mg/capsule | |
|---|---|---|
| Active compound | 20.000 | |
| dl-α-Tocopherol | 0.028 | |
| Hydrogenated Castor Oil | 4.200 | |
| Caprylic/Capric/Stearic Triglyceride | 56.000 | |
| (Synthetic Triglyceride) | | |
| Triglyceride, Medium Chain | 199.772 | |
| Total | 280.000 | mg |

### EXAMPLE 4

### Hard Gelatin Capsules

Hard gelatine capsules were prepared as follows:

TABLE 12
Hard gelatine capsules containing 20-100 mg active substance
20 mg hard gelatine capsule

| Ingredients | mg/capsule | |
|---|---|---|
| Active compound | 20.0 | mg |
| Gelatine Bloom 30 | 70.0 | mg |
| Maltodextrin MD 05 | 108.0 | mg |
| dl-α-Tocopherol | 2.0 | mg |
| Sodium ascorbate | 10.0 | mg |
| Microcrystalline cellulose | 48.0 | mg |
| Magnesium stearate | 2.0 | mg |
| (weight capsule content) | 260.0 | mg |

Procedure: The active substance is wet milled in a solution of gelatine, maltodextrin, dl-α-Tocopherol and sodium ascorbate. The wet milled suspension is spray-dried. The spray-dried powder is mixed with microcrystalline cellulose and magnesium stearate. 260 mg each of this mixture are filled into hard gelatine capsules of suitable size and color.

### EXAMPLE 5

### Tablets

Tablets were prepared as follows:

TABLE 13
Tablets containing 20-50 mg active substance
20 mg tablet

| | mg/tablet | |
|---|---|---|
| Tablet kernel | | |
| Active compound | 20.0 | mg |
| Anhydrous lactose | 130.5 | mg |
| Microcrystalline Cellulose | 80.0 | mg |
| dl-α-Tocopherol | 2.0 | mg |
| Sodium ascorbate | 10.0 | mg |
| Polyvinylpyrrolidone K30 | 5.0 | mg |
| Magnesium stearate | 2.5 | mg |
| (Kernel weight) | 250.0 | mg |
| Film coat | | |
| Hydroxypropyl methylcellulose | 3.5 | mg |
| Polyethylenglycol 6000 | 0.8 | mg |
| Talc | 1.3 | mg |
| Irone oxide, yellow | 0.8 | mg |
| Titanium dioxide | 0.8 | mg |
| (weight of film) | 7.4 | mg |

Procedure: The compound is mixed with anhydrous lactose and microcrystalline cellulose. The mixture is granulated in water with a solution/dispersion of polyvinylpyrrolidone, dl-α-Tocopherol and sodium ascorbate. The granular material is mixed with magnesium stearate and afterwards pressed as kernels with 250 mg weight. The kernels are film coated with a solution/suspension of above-mentioned compositions.

### EXAMPLE 6

### Sachets

Sachets were prepared with the following ingredients:

TABLE 14
Sachets containing 200-500 mg active substance
200 mg sachet

| Ingredients | mg/sachet | |
|---|---|---|
| Active compound | 200.0 | mg |
| Lactose, fine powder | 990.0 | mg |
| Microcrystalline Cellulose | 1250.0 | mg |
| Sodium Carboxymethyl cellulose | 14.0 | mg |
| dl-α-Tocopherol | 5.0 | mg |
| Sodium ascorbate | 20.0 | mg |
| Polyvinylpyrrolidone K30 | 10.0 | mg |
| Magnesium stearate | 10.0 | mg |

All references cited above are incorporated by reference herein as if fully set forth.

I claim:

1. A method of treating multiple sclerosis comprising administering a pharmaceutical preparation comprising a compound selected from the group consisting of a retinoid antagonist, a pharmaceutically acceptable ester or amide thereof and a pharmaceutically acceptable salt of any of these to a patient.

2. The method according to claim 1, wherein the retinoid antagonist is a retinoid RXR antagonist compound selected from the group consisting of
a compound of the formula I, 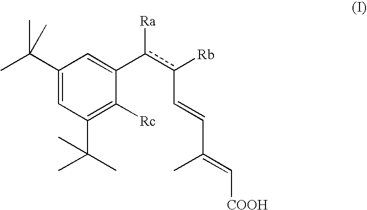
wherein the dotted line is an alternative bond, when the alternative bond is present a double bond exists between the carbon atoms carrying Ra and Rb, Ra is methyl and Rb is hydrogen; when the alternative bond is absent a single, bond exists between the carbon atoms carrying Ra and Rb, Ra and Rb are methylene, and form a cis-substituted cyclopropyl ring with the two carbon atoms carrying Ra and Rb; and Rc is C1-C4-alkoxy;
a compound of the formula II, 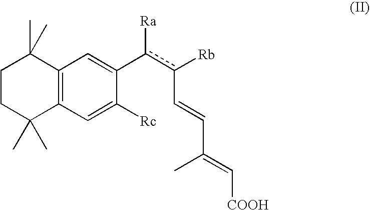
wherein the dotted line is an alternative bond, when the alternative bond is present a double bond exists between the carbon atoms carrying Ra and Rb, Ra is methyl and Rb is hydrogen; when the alternative bond is absent a single, bond exists between the carbon atoms carrying Ra and Rb, Ra and Rb are methylene, and form a cis-substituted cyclopropyl ring with the two carbon atoms carrying Ra and Rb; and Rc is C1-C4-alkoxy;
and a compound of the formula III, 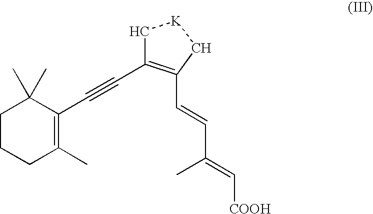
wherein —K— is C1-C4-alkylene, especially —CH2-CH2-CH2-, or is ═CH—CH═ where a benzene ring forms together with the two carbon atoms binding —K—; and Rc is C1-C4-alkoxy;
and in each case a pharmaceutically acceptable amide, ester and/or salt thereof.

3. The method according to claim 1, wherein the retinoid antagonist is an RXR antagonist selected from the group consisting of
(2E,4E,)-(1RS,2RS)-5-[2-(3,5-di-tert-butyl-2-butoxy-phenyl)-cyclopropyl]-3-methyl-penta-2,4-dienoic acid,
(2E,4E,)-(1RS,2RS)-5-[2-(3,5-di-tert-butyl-2-ethoxy-phenyl)-cyclopropyl]-3-methyl-penta-2,4-dienoic acid,
(2E,4E,6Z)-7-[3,5-bis(1,1-dimethylethyl)-2-ethoxyphenyl]-3-methyl-2,4,6-octatrienoic acid ethyl ester,
(2E,4E)-3-methyl-5-[2-(2,6,6-trimethyl-cyclohex-1-enylethynyl)-cyclohept-1-enyl]-penta-2,4-dienoic acid,
(2E,4E)-3-methyl-5-[(1RS,2RS)-2-(5,5,8,8-tetramethyl-3-propoxy-5,6,7,8-tetrahydronaphthalen-2-yl)-cyclopropyl]-penta-2,4-dienoic acid,
(2E,4E,6Z)-3-methyl-7-(5,5,8,8-tetramethyl-3-propoxy-5,6,7,8-tetrahydro-naphthalen-2-yl)-octa-2,4,6-trienoic acid,
(2E,4E,6Z)-7-[2-butoxy-3,5-bis(1,1-dimethylethyl)phenyl]-3-methyl-2,4,6-octatrienoic acid,
and in each case a pharmaceutically acceptable amide, ester and/or salt thereof.

4. The method according to claim 1, wherein one or more symptoms associated with multiple sclerosis are treated or to be treated.

5. The method according to claim 1, wherein the patient is in the stage of primary progressive multiple sclerosis.

6. The method according to claim 1, wherein the patient is in the stage of relapsing-remitting multiple sclerosis.

7. The method according to claim 1, wherein the patient is in the stage of secondary progressive multiple sclerosis.

8. The method according to claim 1, wherein the patient has acute phase multiple sclerosis.

9. The method according to claim 1, wherein the patient is in a stage involving inflammatory processes.

10. (canceled)

11. The method according to claim 1, wherein the retinoid antagonist is a retinoid RXR antagonist; and the pharmaceutical preparation further comprises one or more other pharmaceutically active agents useful in the treatment of multiple sclerosis or one or more of its symptoms.

12. The method according to claim 11, wherein the one or more other pharmaceutically active agents are selected from the group consisting of interferons, interferon β, glatarimer acetate and natalizumab.

13. The method according to claim 1, wherein the pharmaceutical preparation is for oral administration at a daily dosage of about 0.2 mg to about 20 mg of the retinoid antagonist per kg of body weight of the patient.

14. The method according to claim 13, wherein the pharmaceutical preparation is prepared in the form of a tablet, a capsule, a pill or a sachet comprising 10 to 500 mg of the retinoid antagonist.

15. The method according to claim 1, wherein the retinoid antagonist is a retinoid RXR antagonist; and the pharmaceutical preparation further comprises one or more other agents useful against multiple sclerosis.

16. A composition for treatment of multiple sclerosis comprising a retinoid RXR antagonist compound selected from the group consisting of
a compound of the formula I, 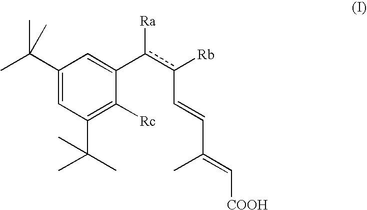
wherein the dotted line is an alternative bond when the alternative bond is present a double bond exists between the carbon atoms carrying Ra and Rb, Ra is methyl and Rb is hydrogen; when the alternative bond is absent a single bond exists between the carbon atoms carrying Ra and Rb, Ra and Rb t are methylene and form a cis-substituted cyclopropyl ring with the two carbon atoms carrying Ra and Rb; and Rc is C1-C4-alkoxy;
a compound of the formula II, 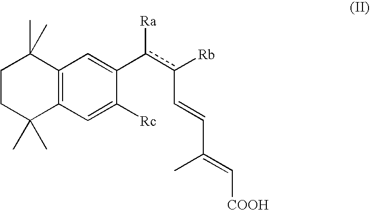
wherein the dotted line is an alternative bond, when the alternative bond is present a double bond exists between the carbon atoms carrying Ra and Rb, Ra is methyl and Rb is hydrogen; when the alternative bond is absent a single bond exists between the carbon atoms carrying Ra and Rb, Ra and Rb are methylene and form a cis-substituted cyclopropyl ring with the two carbon atoms carrying Ra and Rb; and Rc is C1-C4-alkoxy;
and a compound of the formula III, 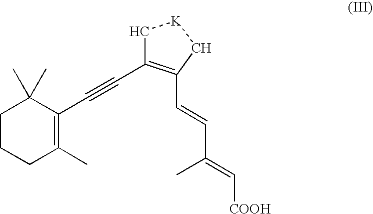
wherein —K— is C1-C4-alkylene, —CH2-CH2-CH2-, or ═CH—CH═ where a benzene ring forms together with the two carbon atoms binding —K—; and Rc is C1-C4-alkoxy;
and in each case a pharmaceutically acceptable amide, ester and/or salt thereof.

17. The composition of claim 16 wherein the multiple sclerosis is in the primary progressive stage.

18. The composition of claim 16 where the multiple sclerosis is in the relapsing-remitting stage.

19. The composition of claim 16, wherein the multiple sclerosis is in the secondary progressive stage.

20. The composition of claim 16, wherein the multiple sclerosis is in the acute phase stage.

21. The composition of claim 16, wherein the involves inflammatory processes.

22. (canceled)

23. A pharmaceutical preparation for the treatment of multiple sclerosis, especially wherever inflammation is one component of the disease manifestations, comprising a retinoid antagonist, a pharmaceutically acceptable ester, a pharmaceutically acceptable amide and/or a pharmaceutically acceptable salt, thereof; and a pharmaceutically acceptable carrier material.

24. The method of claim 11, the retinoid RXR antagonist and the one or more other pharmaceutically active agents are provided separately in a kit and can be combined for simultaneous, separate or sequential administration; and the pharmaceutical preparation is adapted for oral or topical administration.

25. The method of claim 15, the retinoid RXR antagonist and the one or more other pharmaceutically active agents are provided separately in a kit and can be combined for simultaneous, separate or sequential administration; and the pharmaceutical preparation is adapted for oral or topical administration.
